(19) United States
(12) Patent Application Publication
(10) Pub. No.: US 2014/0119610 A1
(43) Pub. Date: May 1, 2014

(54) MEASURING APPARATUS AND METHOD THEREOF

(71) Applicant: GE Medical Systems Global Technology Company, LLC, Waukesha, WI (US)

(72) Inventors: Seiji Funaya, Toyko (JP); Koji Miyama, Tokyo (JP); Yasuyo Saito, Tokyo (JP); Masafumi Ogasawara, Tokyo (JP)

(73) Assignee: GE MEDICAL SYSTEMS GLOBAL TECHNOLOGY COMPANY, LLC, Waukesha, WI (US)

(21) Appl. No.: 14/066,507

(22) Filed: Oct. 29, 2013

(30) Foreign Application Priority Data
Oct. 31, 2012 (JP) 2012-239706

(51) Int. Cl. A61B 8/08; G06T 7/00

(52) U.S. Cl. CPC A61B 8/0891; G06T 7/0016
USPC 382/107

(57) ABSTRACT

A measuring apparatus is provided. The measuring apparatus includes a first region of interest setting unit configured to set first regions of interest to a target for observation, a second region of interest setting unit configured to set a second region of interest to a part not including the first regions of interest, a first moving vector calculating unit configured to calculate moving vectors of first parts of the first regions of interest, a second moving vector calculating unit configured to calculate a moving vector of a second part of the second region, and a corrected moving vector calculating unit configured to perform a correction based on the moving vector of the second part on each of the moving vectors of the first parts to calculate a corrected moving vector.

### CROSS REFERENCE TO RELATED APPLICATIONS

This application claims the benefit of Japanese Patent Application No. 2012-239706 filed Oct. 31, 2012, which is hereby incorporated by reference in its entirety.

### BACKGROUND OF THE INVENTION

The present invention relates to a measuring apparatus which tracks the movement of each of a plurality of parts to which regions of interest set to an ultrasound image are set, to calculate an amount of its movement, and a control program thereof.

The early sensing of a sign of arteriosclerosis is effective in preventing a circulatory system disease such as brain infarction, myocardial infraction or the like. Observing a blood vessel using an ultrasonic diagnostic apparatus has been conducted as a check on the arteriosclerosis. There has been described in, for example, Japanese Unexamined Patent Publication No. 2002-238903 and Japanese Unexamined Patent Publication No. 2010-110373 that the movement of a vascular wall is tracked and measured in an ultrasound image to perform a diagnosis of the arteriosclerosis.

It has been said that when a plaque corresponding to partial hypertrophy due to the arteriosclerosis of a vascular inner wall breaks up, the myocardial infraction or the like is caused. Factors that determine the ease of the breakup of the plaque include the magnitude of a lipid core, the thickness of a fibrous membrane that covers the lipid core, etc. The magnitudes of these lipid cores and the thickness of the fibrous membrane can be estimated by observing the manner of movement of the inside of the plaque. There has therefore been disclosed in a Patent Document 3 that the movements of respective parts in a plaque with the pulsation are tracked to recognize a plaque property of a blood vessel. Specifically, in the Patent Document 3, a region of interest is divided to set divided regions, and the movements of parts to which the divided regions are set are tracked to display moving vectors of the divided regions. The manner of the movement of the inside of the plaque targeted for observation can be recognized by the moving vectors.

A determination as to the property of the plaque will specifically be explained. When the amount of movement of each divided region and its moving direction are not uniform, the plaque is estimated to be soft. On the other hand, when they are uniform, the plaque is estimated to be hard. According to the Patent Document 3, in order to detect the movement (deformation) of the inside of the plaque due to the pulsation, the movement of the part to which each divided region is set, is tracked and the amount of its movement is measured to perform a vector indication. It is therefore possible to recognize the amount of movement of each divided region and its moving direction and thereby determine the property of the plaque.

Thus, recognizing the movement of the inside of the plaque due to the pulsation is useful for determining the property of the plaque. The blood vessel however performs translational motion due to the inertia force with the movement of blood being a viscoelastic material within the blood vessel, the operation of urging an ultrasonic probe on a body surface, and the like. Accordingly, the moving distances obtained by performing the tracking of the divided regions also include factors for movement by the translational motion in addition to factors for movement due to the pulsation. Therefore, in order to eliminate the factors for the movement by the translational motion, there has been described in Japanese Unexamined Patent Publication No. 2012-90819 that the average of the amounts of movement of the divided regions is calculated as the amount of movement of all regions of interest, and a vector of each divided region is represented in the amount of movement from which the average is subtracted (see paragraph 0049 of page 9 in Japanese Unexamined Patent Publication No. 2012-90819).

Since, however, the factors for the movement due to the pulsation are also included in the moving distances calculated by tracking the divided regions, the factors for the movement of the inside of the plaque due to the pulsation, corresponding to information desired to know essentially are also included in the average of the amounts of the movement of the divided regions. Thus, when the average of the amounts of the movement of the divided regions is subtracted, even the factors for the movement due to the pulsation, desired to know essentially are subtracted, thus causing a fear that the motion information due to the pulsation cannot be obtained accurately. There has therefore been a demand for a measuring apparatus capable of more accurately eliminating moving factors other than motion information one desires to know about a target for observation and obtaining more accurate information as motion information desired to know.

### BRIEF DESCRIPTION OF THE INVENTION

In one aspect, a measuring apparatus is provided. The measuring apparatus is equipped with a first region of interest setting unit which sets first regions of interest to a target for observation in an ultrasound image of a subject, a second region of interest setting unit which sets a second region of interest to a part not including the first regions of interest in the ultrasound image, a first moving vector calculating unit which calculates moving vectors of first parts to which the first regions of interest are set, between an ultrasound image at one time phase and an ultrasound image at the other time phase, a second moving vector calculating unit which calculates a moving vector of a second part to which the second region of interest is set, between the ultrasound image at the one time phase and the ultrasound image at the other time phase, and a corrected moving vector calculating unit which performs a correction based on the moving vector of the second part on each of the moving vectors of the first parts to calculate a corrected moving vector.

In another aspect, a measuring apparatus is provided. The measuring apparatus is equipped with a first region of interest setting unit which sets first regions of interest to a target for observation in an ultrasound image of a subject, a second region of interest setting unit which sets a second region of interest to a part not including the first regions of interest in the ultrasound image, a second moving vector calculating unit which calculates a moving vector of a second part to which the second region of interest is set, between an ultrasound image at one time phase and an ultrasound image at the other time phase, and a first moving vector calculating unit which calculates moving vectors of first parts to which the first regions of interest are set, between a position-corrected ultrasound image in which the ultrasound image at the other time phase is position-corrected by a reverse vector of the moving vector of the second part, and the ultrasound image at the one time phase.

According to the one aspect, the second region of interest does not include the first regions of interest set to the target for observation. The moving vector of each of the first parts to which the first regions of interest are set is corrected by the moving vector of the second part to which the second region of interest is set. It is therefore possible to accurately eliminate moving factors other than motion information one desires to know about the target for observation and obtain more accurate information as motion information desired to know.

According to the aspect, the second region of interest does not include the first regions of interest set to the target for observation. The position-corrected ultrasound image in which the ultrasound image at the other time phase is position-corrected by the moving vector of the second part to which the second region of interest is set, is an image from which moving factors other than motion information one desires to know about a target for observation are accurately eliminated. The moving vectors of the first parts to which the first regions of interest are set, are calculated between the position-corrected ultrasound image and the ultrasound image at the one time phase. It is therefore possible to more accurately eliminate moving factors other than motion information one desires to know about a target for observation and obtain more accurate information as motion information desired to know.

Further advantages will be apparent from the following description of exemplary embodiments as illustrated in the accompanying drawings.

### BRIEF DESCRIPTION OF THE DRAWINGS

FIG. 1 is a block diagram showing one example of a schematic configuration of an ultrasonic diagnostic apparatus according to a first embodiment.

FIG. 2 is a block diagram of functions executed by a controller of the first embodiment.

FIG. 3 is a flowchart showing one example of the operation of the ultrasonic diagnostic apparatus of the first embodiment.

FIG. 4 is a diagram illustrating a B-mode image to which first and second regions of interest are set.

FIG. 5 is a conceptual diagram showing B-mode images at time phases T1 and T2.

FIGS. 6A and 6B are diagrams illustrating a B-mode image at a time phase T1 and a B-mode image at a time phase T2, both of which are displayed on a display unit.

FIG. 7 is a diagram for describing the movement of a designated region targeted for observation.

FIG. 8 is a diagram for describing the calculation of a corrected moving vector.

FIG. 9 is a diagram showing a vector indication of corrected moving vectors.

FIG. 10 is a diagram for describing the setting of a second region of interest in a modification of the first embodiment.

FIG. 11 is a block diagram of functions executed by a controller in a second embodiment.

FIG. 12 is a flowchart showing one example of the operation of an ultrasonic diagnostic apparatus according to the second embodiment.

FIG. 13 is a diagram for describing the generation of a position-corrected B-mode image.

FIG. 14 is a diagram showing a B-mode image to which two second regions of interest are set.

### DETAILED DESCRIPTION OF THE INVENTION

Exemplary embodiments will hereinafter be described in detail.

### First Embodiment

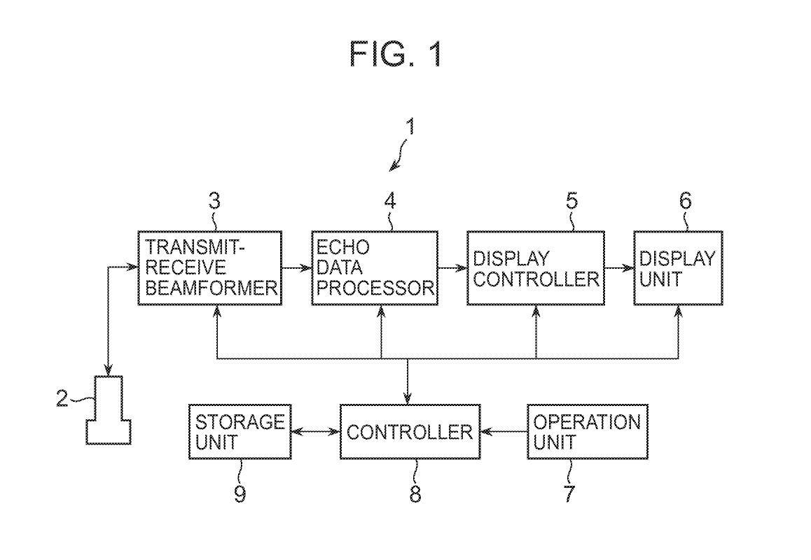

A first exemplary embodiment will first be described based on FIGS. 1 through 8. An ultrasonic diagnostic apparatus 1 shown in FIG. 1 is equipped with an ultrasonic probe 2, a transmit-receive beamformer 3, an echo data processor 4, a display controller 5, a display unit 6, an operation unit 7, a controller 8 and a storage unit 9. The ultrasonic diagnostic apparatus 1 is one example illustrative of an embodiment of a measuring apparatus.

The ultrasonic probe 2 includes a plurality of ultrasonic transducers (not shown) arranged in array form. The ultrasonic probe 2 transmits ultrasound to a subject through the ultrasonic transducers and receives its echo signals therein.

The transmit-receive beamformer 3 supplies an electric signal for transmitting ultrasound from the ultrasonic probe 2 under a predetermined scan condition to the ultrasonic probe 2, based on a control signal outputted from the controller 8. Also, the transmit-receive beamformer 3 performs signal processing such as A/D conversion, phasing-adding processing and the like on each echo signal received by the ultrasonic probe 2 and outputs echo data subsequent to the signal processing to the echo data processor 4.

The echo data processor 4 performs signal processing for generating an ultrasound image on the echo data outputted from the transmit-receive beamformer 3. For example, the echo data processor 4 performs B-mode processing including logarithmic compression processing and envelope detection processing or the like to generate B-mode data.

The display controller 5 performs scan conversion based on a scan converter on the B-mode data to generate B-mode image data. The display controller 5 causes the display unit 6 to display a B-mode image based on the B-mode image data. Also, the display controller 5 causes the display unit 6 to display other indications such as a vector indication vi or the like to be described later.

The display unit 6 includes an LCD (Liquid Crystal Display), a CRT (Cathode Ray Tube) or the like. The operation unit includes a keyboard and a pointing device (not shown) or the like for inputting instructions and information by an operator.

The controller 8 is a CPU (Central Processing Unit) and reads a control program stored in the storage unit 9 to execute functions at the respective parts of the ultrasonic diagnostic apparatus 1. For example, the functions of the transmit-receive beamformer 3, the echo data processor 4 and the display controller 5 may be executed by the control program.

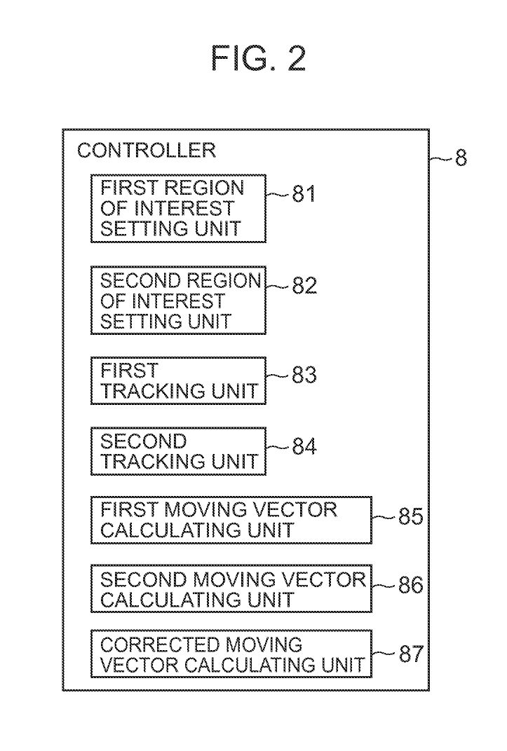

Further, the controller 8 causes the functions of a first region of interest setting unit 81, a second region of interest setting unit 82, a first tracking unit 83, a second tracking unit 84, a first moving vector calculating unit 85, a second moving vector calculating unit 86 and a corrected moving vector calculating unit 87 shown in FIG. 2 to be executed. The details thereof will be described later.

The storage unit 9 is, for example, an HDD (Hard Disk Drive), a semiconductor memory or the like.

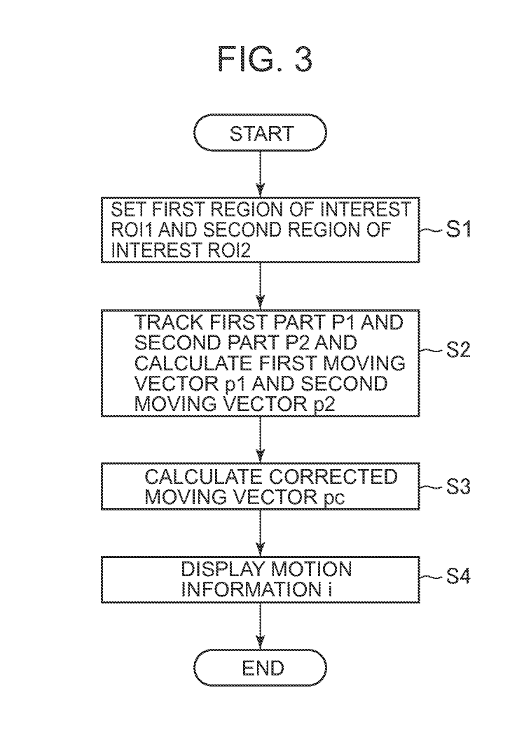

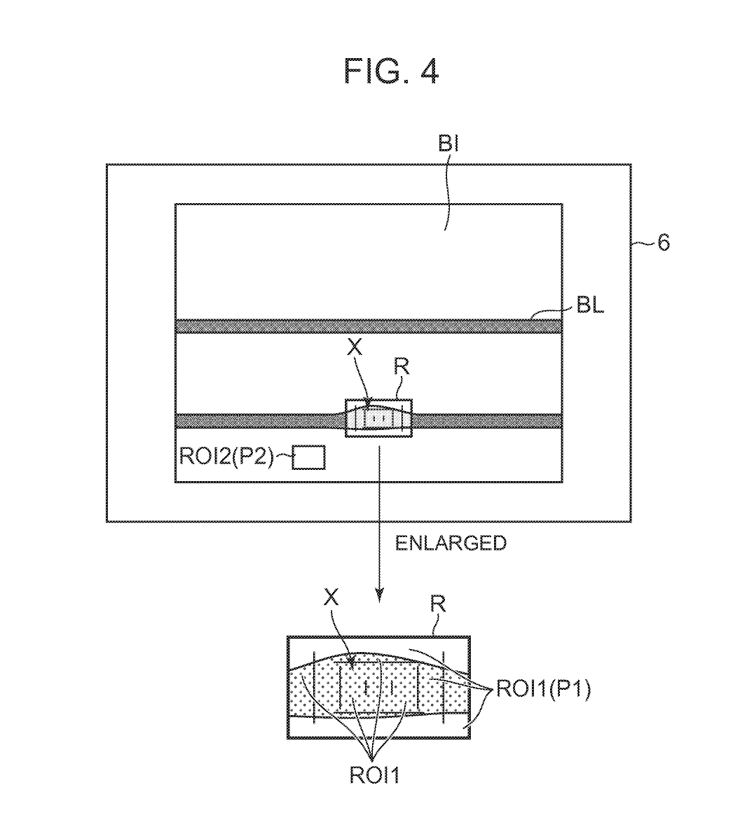

Now, the operation of the ultrasonic diagnostic apparatus 1 according to the first embodiment will be explained based on the flowchart of FIG. 3. First, at Step S1, as shown in FIG. 4, the operator sets first regions of interest ROI1 and a second region of interest ROI2 to a B-mode image BI displayed on the display unit 6 based on echo signals obtained by transmission/reception of ultrasound to and from the subject. The first regions of interest ROI1 and the second region of interest ROI2 are set to a frozen B-mode image BI. Incidentally, this B-mode image BI is an image of a major-axis section of a blood vessel BL.

The setting of the first regions of interest ROI1 will be explained. The first regions of interest ROI1 are regions obtained by dividing a designated region R targeted for observation. When the operator first performs the input of designating a region including a plaque X targeted for observation on the B-mode image using the operation unit 7, the first region of interest setting unit 81 sets an observation-targeted designated region R to the designated region. Next, when the operator performs the input of dividing the observation-targeted designated region R using the operation unit 7, the first region of interest setting unit 81 divides the observation-targeted designated region R and thereby sets the first regions of interest ROI1. The number of divisions of the observation-targeted designated region R may be designated by the operator. In this case, the operator may as well be able to designate the number of divisions by inputting the vertical and horizontal number of divisions.

When the operator performs the input of designating a prescribed region on the B-mode image BI using the operation unit 7, the second region of interest ROI2 is set to the prescribed region by the second region of interest setting unit 82. For example, the second region of interest ROI2 is set below the blood vessel BL.

The first regions of interest ROI1 are regions set to observe internal displacements of the plaque X. Further, the second region of interest ROI2 is a region for detecting a moving factor desired to be eliminated other than moving factors (motion information the operator desires to know about the inside of the plaque X) due to the pulsation. Here, the translational motion of the blood vessel BL due to the inertia force or the like with the movement of blood being a viscoelastic material within the blood vessel BL can be detected by the second region of interest ROI2 set below the blood vessel BL.

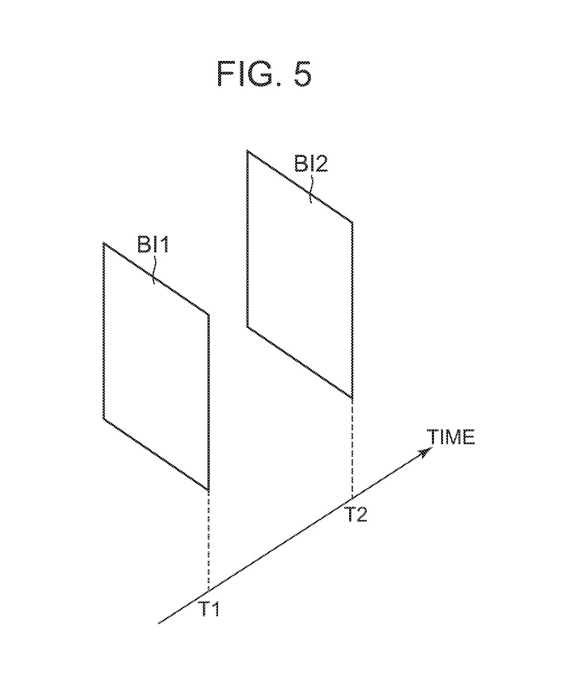

Next, at Step S2, as shown in FIG. 5, the first tracking unit 83 tracks first parts P1 to which the first regions of interest ROI1 are set, between a B-mode image BI1 at a time phase T1 and a B-mode image BI2 at a time phase T2 subsequent to the time phase T1. Further, the second tracking unit 84 tracks a second part P2 to which the second region of interest ROI2 is set, between the B-mode image BI1 and the B-mode image BI2 in like manner. The tracking of the first parts P1 and the second part P2 is performed using, for example, a known method such as an optical flow method or the like.

At Step S2, the first moving vector calculating unit 85 calculates a first moving vector p1 of each of the first parts P1 between the B-mode image BI1 and the B-mode image BI2, based on the result of tracking of each of the first parts P1. The second moving vector calculating unit 86 calculates a second moving vector p2 of the second part P2 between the B-mode image BI1 and the B-mode image BI2, based on the result of tracking of the second part P2.

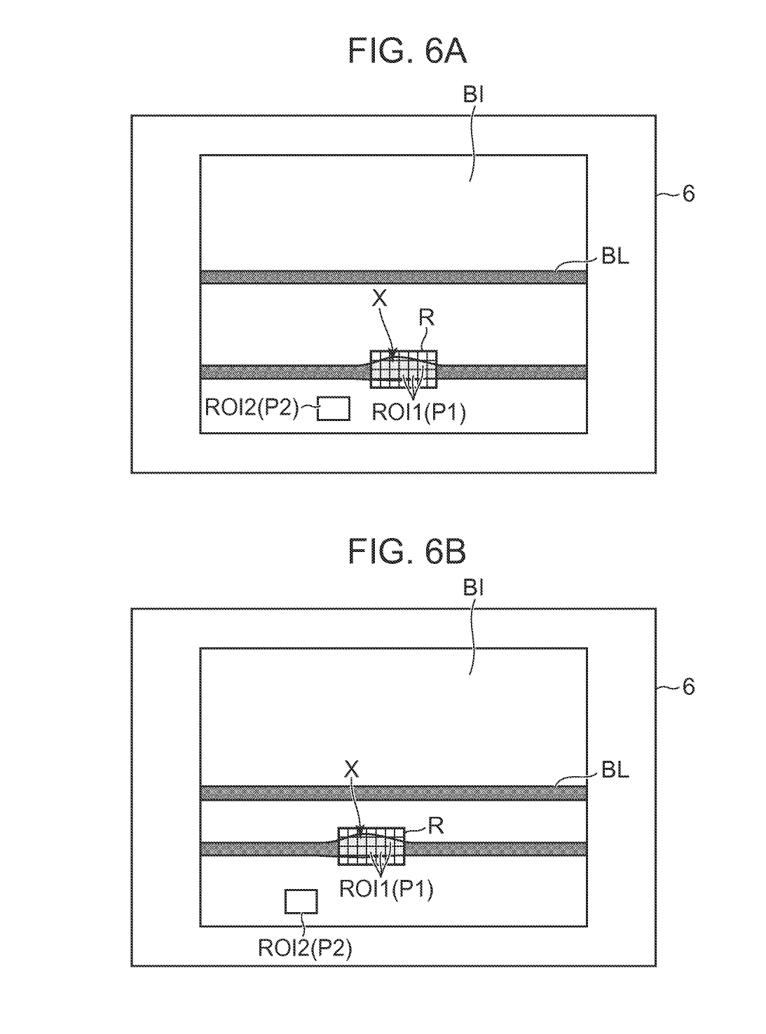

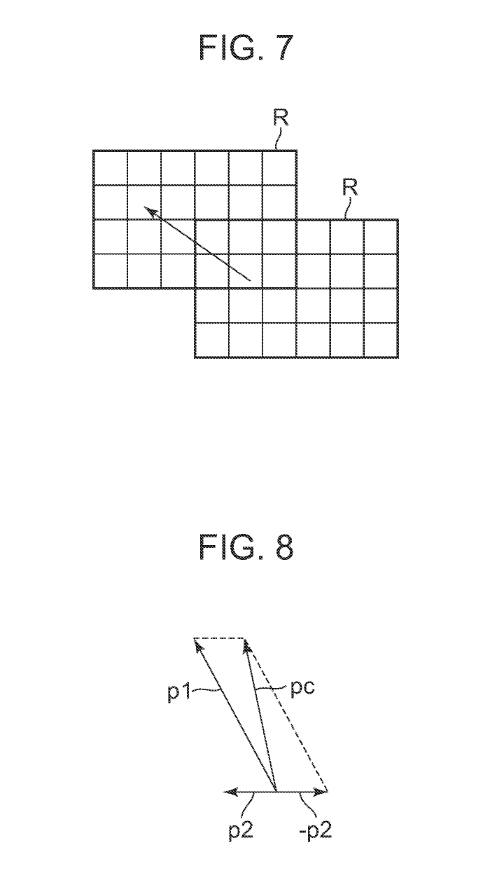

Consider that for example, the ultrasound image B1 at the time phase T1 is an image placed in a state shown in FIG. 6A, and the ultrasound image B2 at the time phase T2 is an image placed in a state shown in FIG. 6B. A blood vessel BL at the ultrasound image B2 is reduced in diameter due to its pulsation with respect to a blood vessel BL at the ultrasound image B1 and further placed in a state of having been translation-moved in the left direction in the figure. Thus, a designated region R targeted for observation is moved in a diagonal upper-left direction as shown in FIG. 7.

Next, at Step S3, the corrected moving vector calculating unit 87 performs a correction based on the second moving vector p2 on each of the first moving vectors p1 and thereby calculates a corrected moving vector pc with respect to each of the first regions of interest ROI1. Described specifically, as shown in FIG. 8, a vector arithmetic operation (subtraction) based on the following Equation 1 is performed to calculate a corrected moving vector pc:

(Corrected moving vector pc)=(the first moving vector p1)−(the second moving vector p2) Equation 1

Although the second moving vector p2 is illustrated as the horizontal vector in FIG. 8, the second moving vector p2 may include a component (not shown) in a minor-axis direction of a blood vessel, where the second part p2 is moved even in the minor-axis direction (vertical direction) of the blood vessel by its pulsation. Accordingly, a corrected moving vector pc from which the factor for the movement in the minor-axis direction due to the pulsation has also been deleted can be obtained.

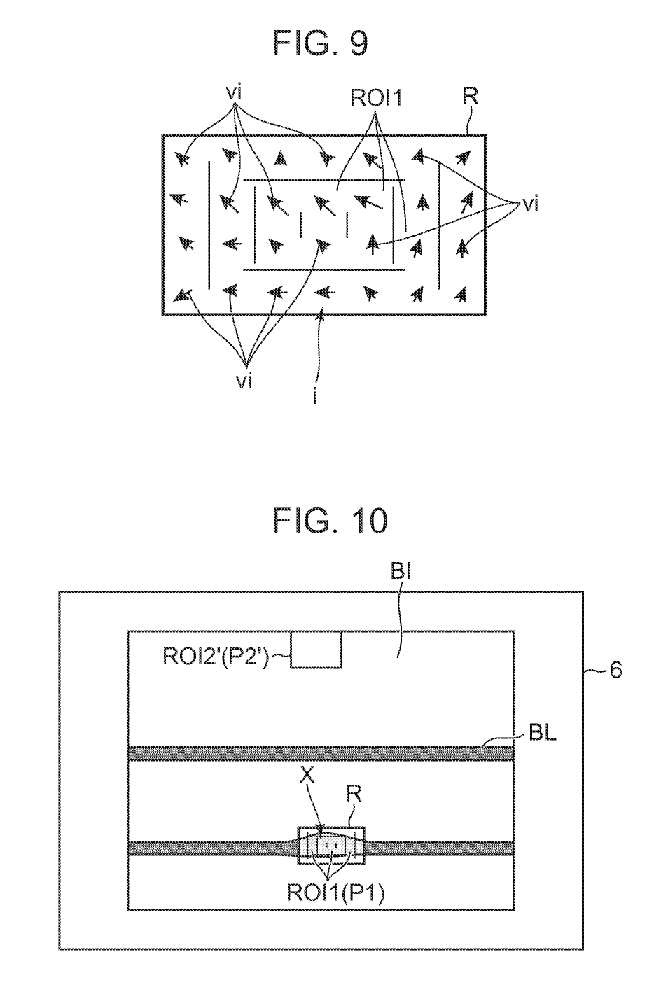

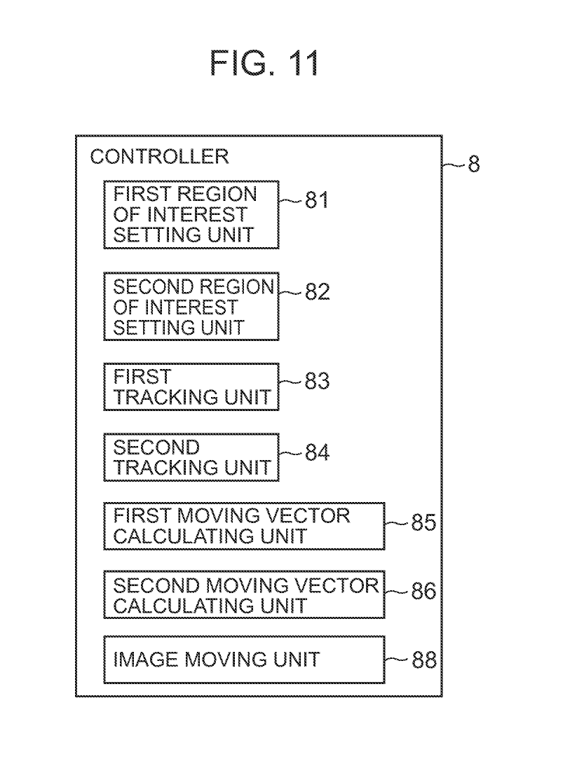

When the corrected moving vector pc is calculated at Step S3, the display controller 5 causes the display unit 6 to display motion information i of each of the first regions of interest ROI1 at Step S4. A vector indication vi indicative of the corrected moving vector pc may be displayed in each of the first regions of interest ROI1 as the motion information i as shown in FIG. 9. This vector indication vi has a display form corresponding to the magnitude of the corrected moving vector pc and its direction. The vector indication vi may be represented aside from the first regions of interest ROI set to the B-mode image BI.

Although not shown, a color (color corresponding to the direction and magnitude of the vector) corresponding to the corrected moving vector pc may be displayed in each of the first regions of interest ROI1 as the motion information i.

According to the first embodiment described above, the second moving vector p2 is subtracted from the first moving vector p1 to calculate the corrected moving vector pc, and the motion information i based on the corrected moving vector pc is displayed. It is therefor possible to display the motion information of each first region of interest ROI from which the factor for the translational motion has been eliminated. Further, since the second moving vector p2 is of the moving vector of the portion (second part P2 to which the second region of interest ROI2 is set) not including the first regions of interest ROI1, it is possible to obtain the motion information from which the moving factors other than the motion information the operator desires to know about the inside of the plaque targeted for observation have been eliminated accurately.

Modifications of the first embodiment will next be explained. The second region of interest setting unit 82 may automatically set a second region of interest ROI2′ to a prescribed position. As shown in FIG. 10, for example, the second region of interest ROI2′ may be set to the vicinity of a body surface of a subject. Thus, the movement of a biological tissue due to the operation of urging the ultrasonic probe 2 on the body surface or the like can be detected by the second region of interest ROI2′ set to the vicinity of the body surface of the subject.

At Step S2 described above, a second part P2′ to which the second region of interest ROI2′ has been set, is tracked between the B-mode image BI1 at the time phase T1 and the B-mode image BI2 at the time phase T2 so that a second moving vector p2 is calculated.

Incidentally, in the unmodified first embodiment, the second region of interest ROI2 may be set to the same position as the first modification, i.e., the vicinity of the body surface of the subject even where the operator sets the second region of interest ROI2 using the operation unit 7.

### Second Embodiment

A second exemplary embodiment will next be described. In the second embodiment, the controller 8 causes the functions of a first region of interest setting unit 81, a second region of interest setting unit 82, a first tracking unit 83, a second tracking unit 84, a first moving vector calculating unit 85, a second moving vector calculating unit 86 and an image moving unit 88 to be executed. The controller 8 in the present embodiment is different from that in the first embodiment and has the image moving unit 88 but does not include the corrected moving vector calculating unit 87.

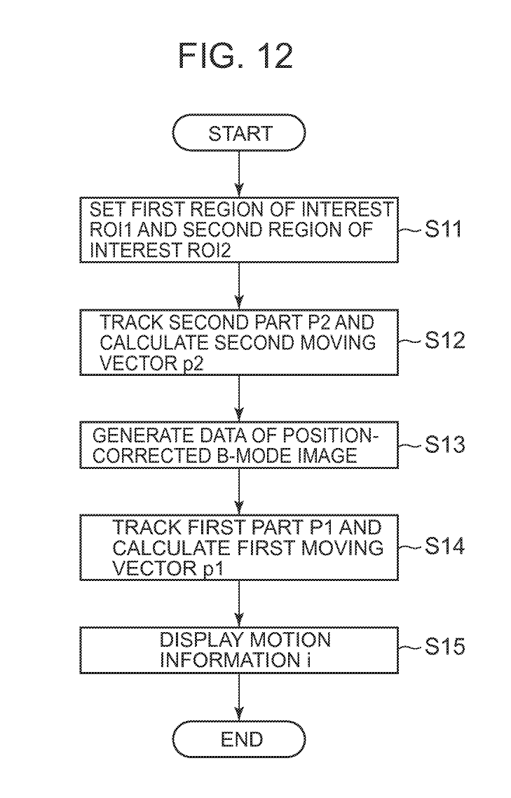

The operation of the ultrasonic diagnostic apparatus 1 according to the second embodiment will next be explained based on the flowchart of FIG. 12. First, at Step S11, the first regions of interest ROI1 and the second region of interest ROI2 are set in a manner similar to Step S1 in the first embodiment.

Next, at Step S12, the second tracking unit 84 tracks a second part P2 to which the second region of interest ROI2 is set, between a B-mode image BI1 at a time phase T1 and a B-mode image BI2 at a time phase T2 subsequent to the time phase T1. Further, the second moving vector calculating unit 86 calculates a second moving vector p2 of the second part P2 between the B-mode image BI1 and the B-mode image BI2, based on the result of tracking of the second part P2.

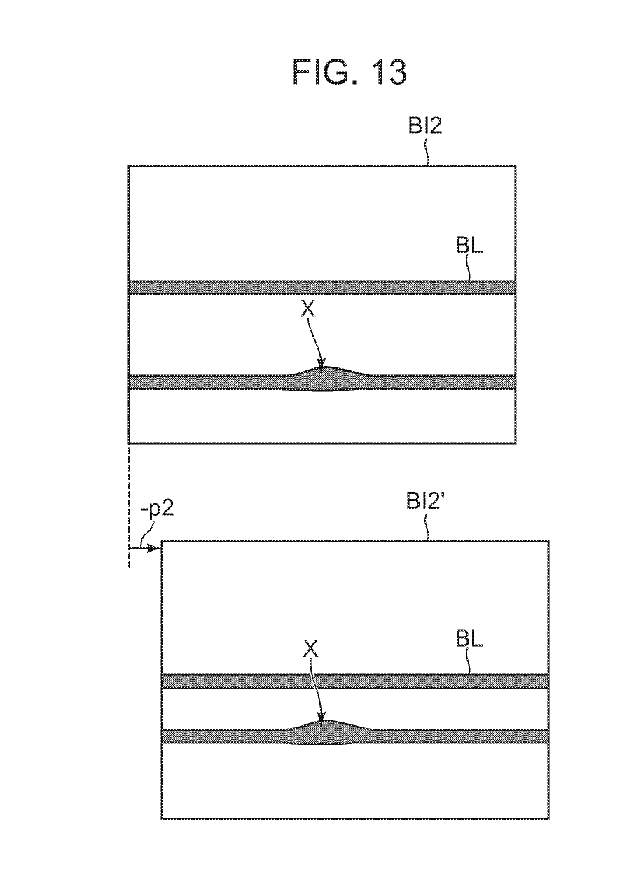

Next, at Step S13, the image moving unit 88 generates data of a position-corrected B-mode image BI2′ in which the B-mode image BI2 is position-corrected by a reverse vector (−p2) of the second moving vector p2, as shown in FIG. 13. Thus, the data of the position-corrected B-mode image BI2′ moved in the reverse direction in the same size as the second moving vector p2 can be obtained with respect to the B-mode image BI2. It is therefore possible to obtain the data of the position-corrected B-mode image BI2′ returned to the position prior to the translational motion. The position-corrected B-mode image BI2′ is an image from which moving factors other than the movement of the inside of a plaque due to pulsation have been eliminated.

Next, at Step S14, the first tracking unit 83 tracks first parts P1 with the first regions of interest ROI1 set thereto, between the B-mode image BI1 at the time phase T1 and the position-corrected B-mode image BI2′. A first moving vector p1 of each of the first parts P1 between the B-mode image BI1 and the position-corrected B-mode image BI2′ is calculated based on the result of tracking of each of the first parts P1.

When the first moving vectors p1 are calculated at Step S14, the display controller 5 causes the display unit 6 to display motion information i of the first regions of interest ROI1 at Step S15. Even in the present embodiment, a vector indication vi (refer to FIG. 8) is displayed as the motion information i as with the first embodiment. The vector indication vi is however an indication representing the size and direction of each of the first moving vectors p1 calculated at Step S14 described above.

Although not illustrated in particular, a color (color corresponding to the direction and magnitude of the vector) corresponding to the first moving vector p1 calculated at Step S14 described above may be displayed in each of the first regions of interest ROI1 as the motion information i.

Incidentally, when each first moving vector p1 between a time phase T2 and a time phase T3 subsequent to the time phase T2 is calculated, the second moving vector calculating unit 86 tracks the second part P2 between the position-corrected B-mode image BI2′ and a B-mode image BI3 at the time phase T3 to calculate a second moving vector p2 at Step S12 described above. A position-corrected B-mode image BI3′ in which the B-mode image BI3 is position-corrected by the second moving vector p2, is obtained. Each of the first parts P1 is tracked between the position-corrected B-mode image BI3′ and the position-corrected B-mode image BI2′ to calculate a first moving vector p1.

According to the second embodiment, the first moving vectors p1 of the first parts P1 with the first regions of interest ROI1 being set thereto are calculated between the position-corrected B-mode image BI2′ in which the translational motion has been canceled, and the B-mode image BI2 at the first time phase. Then, since the motion information i based on the first moving vectors p1 is displayed, it is possible to display motion information of the first regions of interest ROI1 from which the factors for translational motion have been eliminated. Further, since the second moving vector p2 is a moving vector of each part (second part P2 with the second region of interest ROI2 being set thereto) not including the first regions of interest ROI1 as with the first embodiment, it is possible to obtain motion information from which moving factors other than motion information an operator desires to know about the inside of a plaque targeted for observation have been eliminated accurately.

Incidentally, even in the second embodiment, the second region of interest ROI2′ may automatically be set a prescribed position as with the modification of the first embodiment.

Although exemplary embodiments are described herein, it is needless to say that the systems and methods described herein can be changed in various ways within the scope of and without changing the spirit of the invention. For example, in the second embodiment, the second region of interest ROI2 may be set to plural regions. In this case, a position-corrected ultrasound image is generated in which a B-mode image at a time phase T2 has been position-corrected by reverse vectors of moving vectors of a plurality of second parts P2.

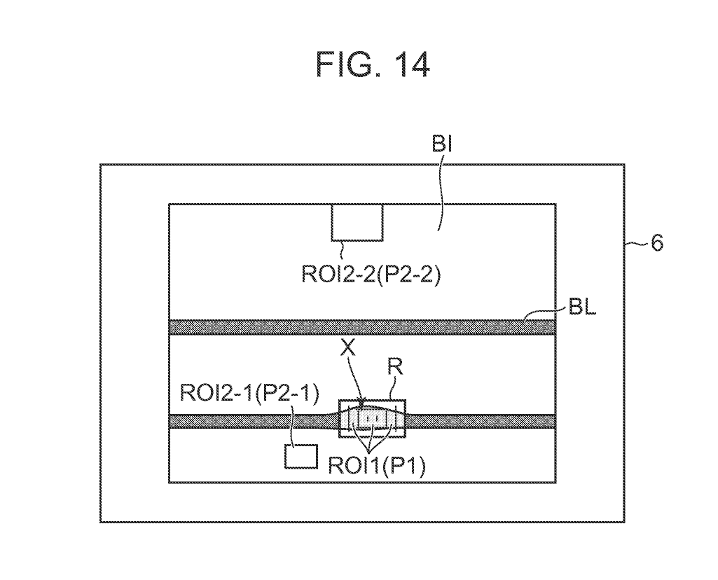

Described specifically, as shown in FIG. 14, both the second regions of interest ROI2-1 and ROI2-2 may be set to below a blood vessel BL and the vicinity of a body surface of a subject in a B-mode image BI. In this case, the second moving vector calculating unit 86 calculates second moving vectors p2-1 and p2-2 of second parts P2-1 and P2-2 to which the respective second regions of interest ROI2-1 and ROI2-2 are set, between a B-mode image BI1 at a time phase T1 and a B-mode image BI2 at a time phase T2, respectively. Then, the second moving vector calculating unit 86 generates a position-corrected B-mode image BI2′ in which the B-mode image BI2 has been position-corrected by the reverse vectors of the second moving vectors p2-1 and further generates a position-corrected B-mode image BI2″ in which the position-corrected B-mode image BI2′ has been position-corrected by the reverse vectors of the second moving vectors p2-2.

When the second regions of interest ROI2-1 and ROI2-2 are set, the second moving vector calculating unit 86 may first calculate second moving vectors p2-2 of second parts P2-2 to which the second regions of interest ROI2-2 have been set, between the B-mode image BI1 at the time phase T1 and the B-mode image BI2 at the time phase T2 and then generate a position-corrected B-mode image BI2′ in which the B-mode image BI2 has been position-corrected by the reverse vectors of the second moving vectors p2-2. In this case, next, the second moving vector calculating unit 86 calculates second moving vectors p2-1 of second parts P2-1 to which the second regions of interest ROI2-1 have been set, between the position-corrected B-mode image BI2′ and the B-mode image BI1, and generates a position-corrected B-mode image BI2″ in which the position-corrected B-mode image BI2′ has been position-corrected by the reverse vectors of the second moving vectors p2-1. In this case, the position-corrected B-mode image BI2′ is one example of illustrative of an embodiment of an ultrasound image at the other time phase.

When the position-corrected B-mode image BI2″ is generated, the tracking of the first parts P1 is performed between the position-corrected B-mode image BI2″ and the B-mode image BI1 at the time phase T1 to calculate the first moving vectors p1.

The measuring apparatus described herein may be implemented in an apparatus other than the ultrasonic diagnostic apparatus. For example, the measuring apparatus described herein may be implemented in a general-purpose computer such as a personal computer. In this case, raw data such as B-mode data or image data such as B-mode image data is fetched into, for example, a general-purpose computer from the ultrasonic diagnostic apparatus, and the processing described in each of the above embodiments is carried out by this general-purpose computer.

Many widely different embodiments may be configured without departing from the spirit and the scope of the present invention. It should be understood that the present invention is not limited to the specific exemplary embodiments described in the specification, except as defined in the appended claims.

1. A measuring apparatus comprising:
a first region of interest setting unit configured to set first regions of interest to a target for observation in an ultrasound image of a subject;
a second region of interest setting unit configured to set a second region of interest to a part not including the first regions of interest in the ultrasound image;
a first moving vector calculating unit configured to calculate moving vectors of first parts of the first regions of interest, between an ultrasound image at a first time phase and an ultrasound image at a second time phase;
a second moving vector calculating unit configured to calculate a moving vector of a second part of the second region, between the ultrasound image at the first time phase and the ultrasound image at the second time phase; and
a corrected moving vector calculating unit configured to perform a correction based on the moving vector of the second part on each of the moving vectors of the first parts to calculate a corrected moving vector.

2. A measuring apparatus comprising:
a first region of interest setting unit configured to set first regions of interest to a target for observation in an ultrasound image of a subject;
a second region of interest setting unit configured to set a second region of interest to a part not including the first regions of interest in the ultrasound image;
a second moving vector calculating unit configured to calculate a moving vector of a second part of the second region of interest, between an ultrasound image at a first time phase and an ultrasound image at another time phase; and
a first moving vector calculating unit configured to calculate moving vectors of first parts of the first regions, between a position-corrected ultrasound image in which the ultrasound image at the second time phase is position-corrected by a reverse vector of the moving vector of the second part, and the ultrasound image at the first time phase.

3. The measuring apparatus of claim 1, wherein the second region of interest setting unit is configured to set the second region of interest to a part for detecting movements away from the target for observation.

4. The measuring apparatus of claim 2, wherein the second region of interest setting unit is configured to set the second region of interest to a part for detecting movements away from the target for observation.

5. The measuring apparatus of claim 1, including a first tracking unit configured to track movements of the first parts, between the ultrasound image at the first time phase and the ultrasound image at the second time phase; and
a second tracking unit configured to track a movement of the second part, between the ultrasound image at the first time phase and the ultrasound image at the second time phase,
wherein the first moving vector calculating unit is configured to calculate the moving vectors of the first parts, based on the tracking of the movements of the first parts by the first tracking unit, and
wherein the second moving vector calculating unit is configured to calculate the moving vector of the second part, based on the tracking of the movement of the second part by the second tracking unit.

6. The measuring apparatus of claim 2, including a first tracking unit configured to track movements of first parts, between the position-corrected ultrasound image and the ultrasound image at the first time phase, and
a second tracking unit configured to track a movement of a second part, between the ultrasound image at the first time phase and the ultrasound image at the second time phase,
wherein the second moving vector calculating unit is configured to calculate the moving vector of the second part, based on the tracking of the movement of the second part by the second tracking unit, and
wherein the first moving vector calculating unit is configured to calculate the moving vectors of the first parts, based on the tracking of the movements of the first parts by the first tracking unit.

7. The measuring apparatus of claim 1, including a display unit configured to display motion information having a display form corresponding to a magnitude and a direction of the corrected moving vector as motion information of each of the first regions of interest.

8. The measuring apparatus of claim 4, including a display unit configured to display motion information having a display form corresponding to a magnitude and a direction of the corrected moving vector as motion information of each of the first regions of interest.

9. The measuring apparatus of claim 2, including a display unit configured to display motion information having a display form corresponding to a magnitude and a direction of the moving vector of each of the first parts as motion information of each of the first regions of interest.

10. The measuring apparatus of claim 5, including a display unit configured to display motion information having a display form corresponding to a magnitude and a direction of the moving vector of each of the first parts as motion information of each of the first regions of interest.

11. A method of measuring movement of a region, comprising:
setting first regions of interest to a target for observation in an ultrasound image of a subject;
setting a second region of interest to a part not including the first regions of interest in the ultrasound image;
calculating moving vectors of first parts of the first regions of interest, between an ultrasound image at a first time phase and an ultrasound image at a second time phase;
calculating a moving vector of a second part of the second region of interest, between the ultrasound image at the first time phase and the ultrasound image at the second time phase; and
performing a correction based on the moving vector of the second part on each of the moving vectors of the first parts to calculate a corrected moving vector.

12. A method of measuring movement of a region, comprising:
setting first regions of interest to a target for observation in an ultrasound image of a subject;
setting a second region of interest to a part not including the first regions of interest in the ultrasound image;
calculating a moving vector of a second part of the second region of interest, between an ultrasound image at a first time phase and an ultrasound image at a second time phase; and
calculating moving vectors of first parts of the first regions of interest, between a position-corrected ultrasound image in which the ultrasound image at the second time phase is position-corrected by a reverse vector of the moving vector of the second part, and the ultrasound image at the first time phase.

13. The method of claim 11, wherein the second region of interest is set to a part for detecting movements away from the target for observation.

14. The method of claim 12, wherein the second region of interest is set to a part for detecting movements away from the target for observation.

15. The method of claim 11, further comprising displaying motion information having a display form corresponding to a magnitude and a direction of the corrected moving vector as motion information of each of the first regions of interest.

16. The method of claim 12, further comprising displaying motion information having a display form corresponding to a magnitude and a direction of the corrected moving vector as motion information of each of the first regions of interest.

17. The measuring apparatus of claim 1, wherein the target for observation is plaque.

18. The measuring apparatus of claim 2, wherein the target for observation is plaque.

19. The method of claim 11, wherein the target for observation is plaque.

20. The method of claim 12, wherein the target for observation is plaque.
